(19) United States
(12) Patent Application Publication
(10) Pub. No.: US 2010/0087383 A1
(43) Pub. Date: Apr. 8, 2010

(54) INTRACEREBRAL OXIDATION INHIBITOR AND USE THEREOF

(75) Inventors: Atsumi Nitta, Aichi (JP); Toshitaka Nabeshima, Aichi (JP)

Correspondence Address: SUGHRUE MION, PLLC, 2100 PENNSYLVANIA AVENUE, N.W., SUITE 800, WASHINGTON, DC, 20037, US

(73) Assignees: National University Corporation Nagoya University, Aichi (JP); Kyowa Hakko Bio Co., Ltd., Tokyo (JP)

(21) Appl. No.: 12/446,893

(22) PCT Filed: Oct. 23, 2007
(86) PCT No.: PCT/JP2007/070628

§ 371 Date: Apr. 23, 2009

(30) Foreign Application Priority Data
Oct. 23, 2006 (JP) 2006-287639

(51) Int. Cl. A61K 38/05; A61P 25/00

(52) U.S. Cl. 514/19

(57) ABSTRACT

The present invention relates to an intracerebral oxidation inhibitor for preventing the onset of mental deterioration, which has early therapeutic effects. Specifically, the present invention relates to: an intracerebral oxidation inhibitor comprising a peptide consisting of Leu and Ile or a modified form thereof as an active ingredient; and a prophylactic or therapeutic agent for mental deterioration comprising the peptide consisting of Leu and Ile or a modified form thereof as an active ingredient and having effects of inhibiting intracerebral oxidation.

### TECHNICAL FIELD

The present invention relates to an intracerebral oxidation inhibitor, use thereof and the like, which are expected to be effective for memory impairment.

### BACKGROUND ART

Acetylcholine esterase inhibitors such as Donepezil are generally used as therapeutic drugs for senile mental deterioration that develops with aging. Because of their strong side effects such as gastrointestinal injuries, application thereof to the elderly is restricted. Furthermore, the effects thereof are to inhibit the progression of mental deterioration, but do not ameliorate mental deterioration. Therefore, a depressor for senile mental deterioration based on a mechanism other than acetylcholine esterase inhibition has been desired.

A cause of memory impairment or the like associated with aging includes brain oxidation. Vitamin E, a lipid-soluble antioxidant that is easily transferred into the central nervous system, is known to have effects of inhibiting the progression of senile mental deterioration (Sano et al., New. Eng. J. Med., 336: 1216-1222 (1997) and An. NY. Acad. Sci. 1031, 249-262 (2004)). However, lipid-soluble antioxidants are insoluble in water and thus are processed with difficulty, and highly concentrated powders cannot be produced with the use of such lipid-soluble antioxidants. Therefore, it is difficult to conveniently produce infusion solutions, tablets, or the like using the same. Furthermore, vitamin E also has side effects such as gastrointestinal injuries, so that a long-term administration thereof to the elderly needs doctor's supervision.

It is known that a dipeptide, Leu-Ile, has therapeutic effects on drug dependence (Nitta A et al., J Neurosci. Res., 78: 250-258 (2004) and Nitta A et al., Folia Pharmacologica Japonica, 122: 81-83 (2003)) and effects of activating Akt involved in a glial cell line-derived neurotrophic factor (GDNF) that suppresses drug dependence (WO/2006/090555). However, it is unknown whether or not Leu-Ile would have effects of inhibiting intracerebral oxidation.

### DISCLOSURE OF THE INVENTION

### Object to be Attained by the Invention

An object of the present invention is to prevent the development of mental deterioration that rapidly increases in the future owing to the aging of the population and to provide an intracerebral oxidation inhibitor having early therapeutic effects.

### Means for Attaining the Object

To achieve the above object, the present inventors have intensively studied the pharmacologic effects of the dipeptide Leu-Ile on a memory impairment model (having developed memory impairment), where the dipeptide significantly differs from vitamin E, that is a lipid-soluble vitamin, in terms of physical property and chemical structure. As a result, the present inventors have discovered that nitration caused by protein oxidation in the hippocampal region is suppressed in an Alzheimer's disease mouse model subjected to intraventricular injection of amyloid protein Aβ 25-35. The present inventors have further discovered that Leu-Ile suppresses memory impairment due to amyloid protein Aβ25-35 in the Alzheimer's disease mouse model.

The present invention is achieved by the above findings and constituted as follows.

(1) An intracerebral oxidation inhibitor comprising a peptide consisting of Leu and Ile or a modified form thereof as an active ingredient.
(2) The intracerebral oxidation inhibitor according to (1), wherein the peptide consisting of Leu and Ile is Leu-Ile.
(3) The intracerebral oxidation inhibitor according to (1), comprising Leu-Ile as an active ingredient.
(4) The intracerebral oxidation inhibitor according to any one of (1) to (3), which inhibits intracerebral oxidation due to an increased amyloid protein level.
(5) The intracerebral oxidation inhibitor according to (4), wherein intracerebral oxidation is the nitration of intracerebral protein.
(6) A prophylactic or therapeutic agent for mental deterioration comprising a peptide consisting of Leu and Ile or a modified form thereof as an active ingredient wherein the agent has an effect of inhibiting intracerebral oxidation.
(7) The prophylactic or therapeutic agent for mental deterioration according to (6), wherein the peptide consisting of Leu and Ile is Leu-Ile.
(8) The prophylactic or therapeutic agent for mental deterioration according to (6), wherein the active ingredient is Leu-Ile.
(9) The prophylactic or therapeutic agent for mental deterioration according to any one of (6) to (8), which is used for mental deterioration due to an increased amyloid protein level.

In the description, peptides are denoted according to conventional notation such that the left end is the amino terminus and the right end is the carboxyl terminus. Moreover, even when an amino acid residue is an L-amino acid residue, related notation (“L-”) is abbreviated.

The present inventors further provide the following inventions based on their own pharmacological findings.

(10) Use of a peptide consisting of Leu and Ile or a modified form thereof in manufacture of an intracerebral oxidation inhibitor.
(11) Use of a peptide consisting of Leu and Ile or a modified form thereof in manufacture of a prophylactic or therapeutic agent for mental deterioration, wherein the agent has an effect of inhibiting intracerebral oxidation.
(12) A prophylactic or therapeutic agent for mental deterioration, comprising a peptide consisting of Leu and Ile or a modified form thereof as an active ingredient.
(13) The prophylactic or therapeutic agent for mental deterioration according to (12), wherein the peptide consisting of Leu and Ile is Leu-Ile.
(14) The prophylactic or therapeutic agent for mental deterioration according to (12), wherein the active ingredient is Leu-Ile.
(15) The prophylactic or therapeutic agent for mental deterioration according to any one of (12) to (14), wherein mental deterioration is Alzheimer's disease.
(16) A method for preventing or treating mental deterioration, using a peptide consisting of Leu and Ile or a modified form thereof.
(17) The method for preventing or treating mental deterioration according to (16), wherein the peptide consisting of Leu and Ile is Leu-Ile.
(18) The method for preventing or treating mental deterioration according to (16), wherein Leu-Ile is used.
(19) The method for preventing or treating mental deterioration according to any one of (16) to (18), wherein mental deterioration is Alzheimer's disease and a mild cognitive impairment (hereinafter, referred to as “MCI”).

### EFFECTS OF THE INVENTION

According to the present invention, an intracerebral oxidation inhibitor inhibiting intracerebral protein oxidation caused by aging or a prophylactic or therapeutic agent for mental deterioration caused by aging is provided.

### BRIEF DESCRIPTION OF THE DRAWINGS

FIG. 1 is a graph showing the effects of Leu-Ile to inhibit the nitration of intracerebral protein in an Alzheimer's disease mouse model subjected to intraventricular injection of Aβ.

FIG. 2 is a graph showing the effects of oral administration of Leu-Ile to inhibit memory impairment in an Alzheimer's disease mouse model subjected to intraventricular injection of Aβ.

FIG. 3 is a graph showing the effects of intraperitoneal administration of Leu-Ile to inhibit memory impairment in an Alzheimer's disease mouse model subjected to intraventricular injection of Aβ.

This description includes part or all of the contents as disclosed in the description of Japanese Patent Application No. 2006-287639, which is a priority document of the present application.

### BEST MODES FOR CARRYING OUT THE INVENTION

In the present invention, examples of a peptide consisting of Leu and Ile include Leu-Ile and Ile-Leu. Leu-Ile is preferably used.

In the present invention, the term “a peptide consisting of Leu and Ile or a modified form thereof” refers to a compound having a basic structure differing at least partially from the basic structure consisting of Leu and Ile (dipeptide) because of modification such as the substitution of a portion (or a plurality of portions) with another atomic group(s) or addition of another molecule(s), for example. Furthermore, a dipeptide in which at least one of Leu and Ile has been substituted with D- or DL-form thereof is also an example of a peptide consisting of Leu and Ile or a modified form thereof.

A typical example of such modified form in the present invention includes a peptide derivative prepared by substituting a portion of a side chain of Leu or Ile with another atom or an atomic group. Examples of another atom or atomic group include a hydroxyl group, halogen (e.g., fluorine, chlorine, bromine, and iodine), an alkyl group (e.g., a methyl group, an ethyl group, an n-propyl group, and an isopropyl group), a hydroxyalkyl group (e.g., a hydroxymethyl group and a hydroxyethyl group), an alkoxy group (e.g., a methoxy group and an ethoxy group), and an acyl group (e.g., a formyl group, an acetyl group, a malonyl group, and a benzoyl group).

Examples of a modified form of the peptide of the present invention also include a modified peptide in which a functional group of Leu or Ile is protected by an appropriate protecting group. Examples of a protecting group that can be used for such purpose include an acyl group, an alkyl group, a monosaccharide, an oligosaccharide, and a polysaccharide. Such a protecting group is linked via an amide bond, an ester bond, a urethane bond, a urea bond, or the like according to a peptide site to which a protecting group is bound, the type of a protecting group to be used, and the like.

Further examples of a modified form of the peptide of the present invention include those modified by glycosylation. Examples of a modified form of the peptide of the present invention also include various peptide derivatives prepared by substitution of the N-termini or C-termini with other atoms, for example, which are classified into alkylamines, alkylamides, sulfinyls, sulfonylamides, halides, amides, amino alcohols, esters, amino aldehydes, and the like. In addition, peptide derivatives composed by combining the above-explained various modification techniques may also be examples of a modified form of the peptide of the present invention.

Examples of the peptide consisting of Leu and Ile or a modified form thereof of the present invention also include a salt of the above peptide, a salt of the above modified form of the peptide, or a hydrate thereof. The type of such a salt to be used in the present invention is not particularly limited, as long as it is pharmaceutically acceptable. Examples of such salt include a salt formed with hydrochloric acid, phosphoric acid, sulfuric acid, nitric acid, boric acid, or the like (inorganic acid salt) and a salt formed with formic acid, acetic acid, lactic acid, fumaric acid, maleic acid, tartaric acid, citric acid, or the like (organic acid salt). These salts can be prepared by conventional means.

Examples of an intracerebral oxidation inhibitor in the present invention include oxidation inhibitors containing active ingredients that are known to exhibit the effects of inhibiting oxidation of biomaterials in the central nervous system as a result of oral administration. Examples of the effects of inhibiting oxidation include inhibition of the nitration of intracerebral proteins and elimination of free radicals generated within the brain. Because of such effects of inhibiting intracerebral oxidation, the intracerebral oxidation inhibitor of the present invention can be used for preventing or treating diseases due to the progression of intracerebral oxidation.

Intracerebral oxidation is a phenomenon that takes place caused by stress, allergy, drug poisoning, toxic poisoning, aging, or the like. It is also known that intracerebral oxidation takes place due to an increased intracerebral amyloid protein level, resulting in reduced memory and paralysis of cerebral functions. Therefore, an example of diseases caused by the progression of intracerebral oxidation is mental deterioration. Examples of mental deterioration include MCI, Alzheimer's disease, Pick's disease, Lewy body dementia, and memory impairment in adults or elderly. Examples of such memory impairment include mild or severe memory impairment. An example of severe memory impairment is dementia.

The onset of Alzheimer's disease begins from the 50s because of intracerebral oxidation, amyloid protein deposition in the brain, reduced nutritional supply to the brain, or the like. Symptoms of namely memory impairment become significant with time over 20 or more years in most cases, so that patients are determined to be affected by dementia. A therapeutic agent for mental deterioration such as an acetylcholine esterase inhibitor is administered after determination. Therefore, a substance for preventing the significant progression of mental deterioration, which is administered before such determination, is regarded as a prophylactic agent.

A method for preventing mental deterioration in the present invention also includes the administration of a dietary supplement or a medicament such as OTC for inhibiting mental deterioration symptoms that may be developed in the future.

In the present invention, the peptide consisting of Leu and Ile or a modified form thereof exerts effects when amyloid protein deposition takes place, so that it is used as a prophylactic agent or therapeutic agent for mental deterioration.

The term “mental deterioration” in the present invention refers to mild or severe memory impairment. Examples of mild memory impairment include MCI, memory loss, and amnesia. Examples of severe memory impairment include Alzheimer's disease, Pick's disease, and Lewy body dementia, which are determined to be dementia. Therefore, the term “mental deterioration” in the present invention is not limited to those recognized as dementia.

Examples of an intracerebral oxidation inhibitor and a prophylactic or therapeutic agent for mental deterioration in the present invention include pharmaceutical preparations or dietary supplements. Examples of dietary supplements include preparations for oral administration that have similar shapes of medicaments, but are not classified as medicaments under the Pharmaceutical Law or supplements prepared by adding an active ingredient of the present invention to foods.

Specifically, the present invention relates to a pharmaceutical composition or a nutritional composition for a dietary supplement, which contains the peptide consisting of Leu and Ile or a modified form thereof as an active ingredient.

The term “pharmaceutical preparation” in the present invention refers to a preparation prepared by adding a carrier that is generally used as a base for pharmaceutical preparation, such as an excipient, a disintegrator, a lubricant, a buffering agent, a binder, an emulsifier, a suspension, a soothing agent, a stabilizer, a preservative, an antiseptic, a physiological saline solution, or the like to an active ingredient of the present invention. Examples of formulations include tablets, powders, fine granules, granules, capsules, syrups, injections, external preparations, and suppositories. Examples of an excipient that can be used herein include lactose, starch, sorbitol, D-mannitol, and saccharose. Examples of disintegrators that can be used herein include starch, carboxymethylcellulose, and calcium carbonate. Examples of buffering agents that can be used herein include phosphate, citrate, and acetate. Examples of emulsifiers that can be used herein include gum Arabic, sodium alginate, and tragacanth. Examples of binders that can be used herein include pullulan, gum Arabic, gelatin, and starch. Examples of lubricants that can be used herein include magnesium stearate, methylcellulose, and magnesium silicate. Examples of suspensions that can be used herein include glyceryl monostearate, aluminum monostearate, methylcellulose, carboxymethylcellulose, hydroxymethylcellulose, and sodium lauryl sulfate. Examples of soothing agents that can be used herein include benzyl alcohol, chlorobutanol, and sorbitol. Examples of stabilizers that can be used herein include propylene glycol, diethylin sulfite, and ascorbic acid. Examples of preservatives that can be used herein include phenol, benzalkonium chloride, benzyl alcohol, chlorobutanol, and methylparaben. Examples of antiseptics that can be used herein include benzalkonium chloride, parahydroxybenzoate, and chlorobutanol.

The term “dietary supplement (oral preparation)” in the present invention refers to a preparation prepared by adding a carrier that is generally used as a nutritional food preparation base, such as an excipient, a disintegrator, an emulsifier, a stabilizer, a lubricant, a buffering agent, a flavoring agent, or the like to an active ingredient of the present invention. Examples of formulations include tablets, powders, fine granules, granules, and capsules.

Examples of dietary supplements prepared by adding an active ingredient of the present invention to foods include nutritional beverages, soft drinks, and jelly, which are produced according to a conventional method using an active ingredient of the present invention.

The peptide consisting of Leu and Ile can be produced by a known peptide synthesis method (e.g., a solid phase synthesis method and a liquid phase synthesis method). When the peptide of the present invention and the like are present in the nature, they can also be prepared by procedures such as extraction and purification. Examples of a source for obtainment of the peptide of the present invention and the like include animal cells (including human cells), plant cells, and body fluids (e.g., blood and urine).

Furthermore, an efficient method for producing dipeptides using microorganisms, enzymes, and the like is known (WO2004-058960). The peptide consisting of Leu-Ile can be efficiently produced according to the production method disclosed in the patent publication.

Furthermore, a modified form of the peptide consisting of Leu and Ile can be produced by a conventional method as described above.

A pharmaceutical preparation or an oral dietary supplement preparation containing a modified form of the peptide consisting of Leu and Ile as an active ingredient can be produced according to a conventional method using the obtained modified form of the peptide consisting of Leu and Ile as a major active ingredient.

When formulated, a desired preparation can be produced so that a modified form of the peptide consisting of Leu and Ile contains pharmaceutically acceptable other ingredients (e.g., carriers, excipients, disintegrators, buffering agents, emulsifiers, binders, lubricants, suspensions, soothing agents, stabilizers, preservatives, antiseptics, and physiological saline solutions). Examples of excipients that can be used herein include lactose, starch, sorbitol, D-mannitol, and saccharose. Examples of disintegrators that can be used herein include starch, carboxymethylcellulose, and calcium carbonate. Examples of buffering agents that can be used herein include phosphate, citrate, and acetate. Examples of emulsifiers that can be used herein include gum Arabic, sodium alginate, and tragacanth. Examples of binders that can be used herein include pullulan, gum Arabic, gelatin, and starch. Examples of lubricants that can be used herein include magnesium stearate, methylcellulose, and magnesium silicate. Examples of suspensions that can be used herein include glyceryl monostearate, aluminum monostearate, methylcellulose, carboxymethylcellulose, hydroxymethylcellulose, and sodium lauryl sulfate. Examples of soothing agents that can be used herein include benzyl alcohol, chlorobutanol, and sorbitol. Examples of stabilizers that can be used herein include propylene glycol, diethylin sulfite, and ascorbic acid. Examples of preservatives that can be used herein include phenol, benzalkonium chloride, benzyl alcohol, chlorobutanol, and methylparaben. Examples of antiseptics that can be used herein include benzalkonium chloride, parahydroxybenzoate, and chlorobutanol.

With the use of these preparation bases, desired formulations (e.g., tablets, powders, fine granules, granules, capsules, syrups, injections, external preparations, and suppositories) can be produced by a conventional method.

Nutritional foods such as nutritional beverages, soft drinks, and jelly can be produced by adding a modified form of the peptide consisting of Leu and Ile according to a conventional method.

The thus formulated intracerebral oxidation inhibitor or a prophylactic or therapeutic agent for mental deterioration containing the peptide consisting of Leu and Ile or a modified form thereof of the present invention as an active ingredient can be applied to patients via oral administration or parenteral administration (e.g., intravenous injection, intraarterial injection, subcutaneous injection, intramuscular injection, and intraperitoneal injection) according to their forms. The content of an active ingredient (e.g., peptide) of the present invention in a drug generally differs depending on formulations. In the case of a liquid preparation such as an injection, the content ranges from approximately 0.001% by weight to approximately 90% by weight and ranges from, to achieve a desired dose, approximately 0.001% by weight to approximately 10% by weight, preferably ranges from 0.01% by weight to approximately 3% by weight, and particularly preferably ranges from 0.1% by weight to approximately 1% by weight, for example. In the case of a solid agent such as a tablet, the content ranges from 0.1% by weight to approximately 90% by weight, preferably ranges from 1% by weight to approximately 50% by weight, and particularly preferably ranges from 3% by weight to approximately 30% by weight.

According to the present invention, a method for preventing or a method for treating mental deterioration using a preparation containing the peptide consisting of Leu and Ile or a modified form thereof as an active ingredient is provided. The therapeutic method or the prophylactic method of the present invention comprises a step of administering a preparation containing the peptide consisting of Leu and Ile or a modified form thereof as an active ingredient to a living body. The route of administration is not particularly limited and examples thereof include oral, intravenous, intradermal, subcutaneous, intramuscular, intraperitoneal, transdermal, and transmucosal administration. The dose of a drug differs depending on symptoms, the patient's age, sex, and body weight, and the like. Persons skilled in the art can adequately determine the appropriate dose. For example, when a preparation containing Leu-Ile as an active ingredient is used, the dose can be determined so that the amount of the active ingredient per day for an adult (body weight of approximately 60 kg) ranges from approximately 0.1 (mg) to approximately 3000 mg, preferably ranges from approximately 1 mg to approximately 2000 mg, and particularly preferably ranges from approximately 3 mg to approximately 1000 mg. Administration can be scheduled so that administration is carried out once a day to several times a day, once per two days, or once per three days, for example.

In the present invention, a preparation comprising the peptide consisting of Leu and Ile or a modified form thereof as an active ingredient means prophylactic ingestion of a preparation containing the peptide consisting of Leu and Ile or a modified form thereof as an active ingredient in order to prevent an adult who has not expressed any mental deterioration symptoms from expressing mental deterioration symptoms such as reduced memory due to aging. Further, a method for treating mental deterioration, wherein a preparation comprising the peptide consisting of Leu and Ile or a modified form thereof as an active ingredient is administered, means administration to a patient who has expressed mental deterioration symptoms such as reduced memory in order to inhibit or ameliorate the progress of mental deterioration symptoms such as memory impairment and peripheral mental deterioration symptoms. Administration can be scheduled considering the patient's pathological conditions, the duration of the action of the drug, and the like.

### EXAMPLES

Examples of the present invention are as described below.

### Example 1

### Effects of Leu-Ile to Inhibit Intracerebral Protein Oxidation in Alzheimer's Disease Mouse Model Subjected to Intraventricular Injection of Aβ

An Alzheimer's disease mouse model (Maurice et al. Brain Res. 705, 181-193, 1996) was produced by injecting Aβ25-35 (the aggregation of which had been accelerated by 4 days of incubation at 37° C.) into mouse cerebral ventricle and then used for the experimental system. ICR mice (body weight of 20-22 g (7 weeks old), purchased from Japan SLC Inc. (Shizuoka, Japan)) were divided into 5 groups (n=12-15). A physiological saline solution was injected instead of Aβ25-35 to the cerebral ventricle of the control group. A physiological saline solution that is a solvent for Leu-Ile, Leu-Ile (1.5 μmol/Kg), or Leu-Ile (15 μmol/Kg), was injected into Alzheimer's disease mouse model groups. Then the groups were used for the experiment.

Moreover, an Leu-Ile 1.5 μmol/Kg administrated group and an Leu-Ile 15 μmol/Kg administrated group were subjected to intraperitoneal administration of Leu-Ile (1.5 μmol/Kg) and Leu-Ile (15 μmol/Kg), respectively, once a day, immediately after intraventricular administration of Aβ25-35.

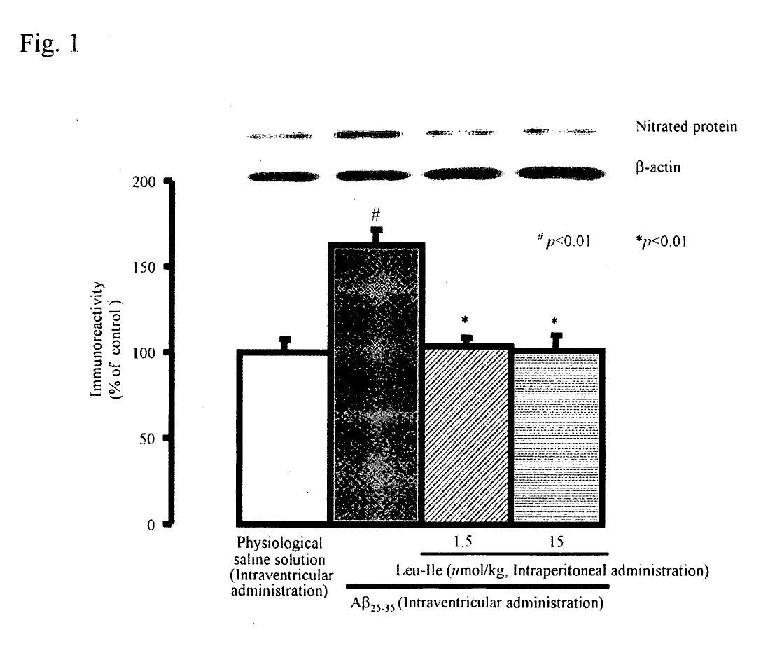

A novel object recognition test was conducted for each group 3 to 5 after intraventricular administration of Aβ25-35 or a solvent. Decapitation was carried out to excise the hippocampus and then protein nitration in the hippocampal region was confirmed by a Western blot method (Tran et al., Mol. Psychy., 8, 407-412, 2003). The results are shown in FIG. 1.

As shown in FIG. 1, whereas the nitration of a 70-KD protein was increased in the hippocampal regions in the control group that had developed Alzheimer compared with the other control group, the nitration of the 70-KD protein in the hippocampal regions was inhibited in both the Leu-Ile 1.5 μmol/Kg administrated group and the Leu-Ile 15 μmol/Kg administrated group. In addition, dose dependence was not observed. Furthermore, when Leu-Ile (15 μmol/Kg) was administered to untreated mice, effects of improving memory were not observed.

### Example 2

### Effects of Oral Administration of Leu-Ile on Learning and Memory Impairment Confirmed by a New Object Identification Test Conducted for an Alzheimer's Disease Mouse Model Subjected to Intraventricular Injection of Aβ

An Alzheimer's disease mouse model was produced by injecting Aβ25-35 (the aggregation of which had been accelerated by 4 days of incubation at 37° C.) to mouse cerebral ventricle, following which it was used for the experimental system. ICR mice (body weight of 20-22 g (7 weeks old), purchased from Japan SLC Inc. (Shizuoka, Japan)) were divided into 4 groups (n=15). A physiological saline solution that is a solvent for Leu-Ile, Leu-Ile (1.5 μmol/Kg), or Leu-Ile (15 μmol/Kg) was administered to Alzheimer's disease mouse model groups. Then the groups were used for the experiment.

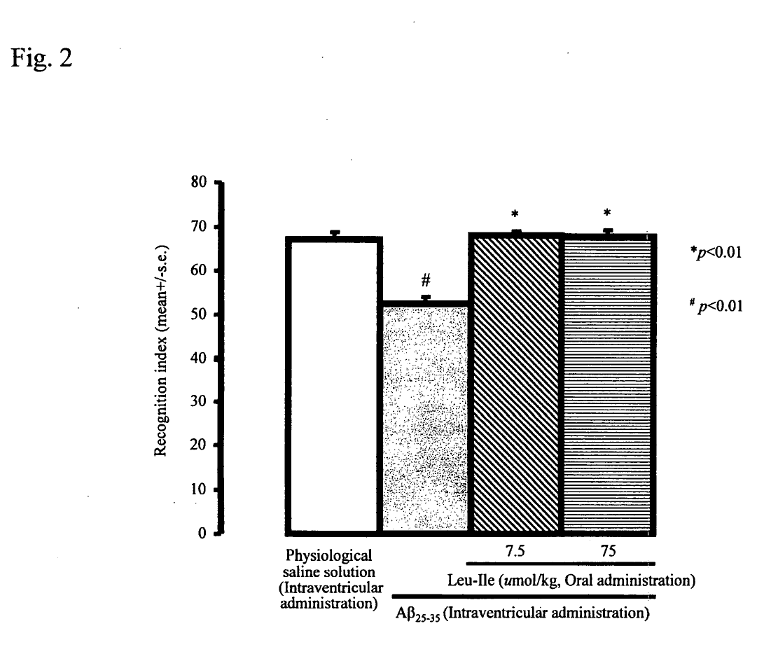

Moreover, an Leu-Ile 7.5 μmol/Kg administrated group and an Leu-Ile 75 μmol/Kg administrated group were subjected to oral administration of Leu-Ile (7.5 μmol/Kg) and Leu-Ile (75 μmol/Kg), respectively, immediately after administration of Aβ25-35. A novel object recognition test (Ennaceur et al., Behav Brain Res 80 9-25, 1996) was conducted for all groups 3 to 5 days after intraventricular administration of Aβ25-35 or a physiological saline solution. The test results on day 5 after administration of Aβ25-35 or a physiological saline solution were compared with the other test results on day 5 after administration (FIG. 2).

As shown in FIG. 2, the control group, which had developed Alzheimer because of the administration of Aβ25-35, exerted significantly decreased recognition indices compared with the control group to which only a physiological saline solution had been administered. However, decreased recognition indices were not confirmed in the Leu-Ile 7.5 μmol/Kg administrated group or in the Leu-Ile 75 μmol/Kg administrated group. It was thus confirmed that Leu-Ile exerts effects of significantly inhibiting the progression of Alzheimer's dementia. In addition, dose dependence was not observed.

### Example 3

### Effects of Oral Administration of Leu-Ile on Learning and Memory Impairment Confirmed by a Novel Object Recognition Test Conducted for an Alzheimer's Disease Mouse Model Subjected to Intraventricular Injection of Aβ

An Alzheimer's disease mouse model was produced by injecting Aβ25-35 (the aggregation of which had been accelerated by 4 days of incubation at 37° C.) to mouse cerebral ventricle and then used for the experimental system. ICR mice (body weight of 20-22 g, purchased from Japan SLC Inc. (Shizuoka, Japan)) were divided into 5 groups (n=15). A physiological saline solution was administered instead of Aβ25-35 to the cerebral ventricle of a control group. A physiological saline solution; that is a solvent for Leu-Ile, Leu-Ile (1.5 μmol/Kg), or Leu-Ile (15 μmol/Kg) was administered to Alzheimer's disease mouse model groups. Then the groups were used for the experiment.

The Alzheimer's disease model control group, the Leu-Ile 1.5 μmol/Kg administrated group, the Leu-Ile 15 μmol/Kg administrated group, and an Leu-Ile 75 μmol/Kg administrated group were separately subjected to the experiment.

Specifically, the Leu-Ile 7.5 μmol/Kg administrated group, the Leu-Ile 15 μmol/Kg administrated group, and the Leu-Ile 75 μmol/Kg administrated group were subjected to intraperitoneal administration of Leu-Ile (7.5 μmol/Kg), Leu-Ile (15 μmol/Kg), and Leu-Ile (75 μmol/Kg), respectively, immediately after administration of Aβ25-35.

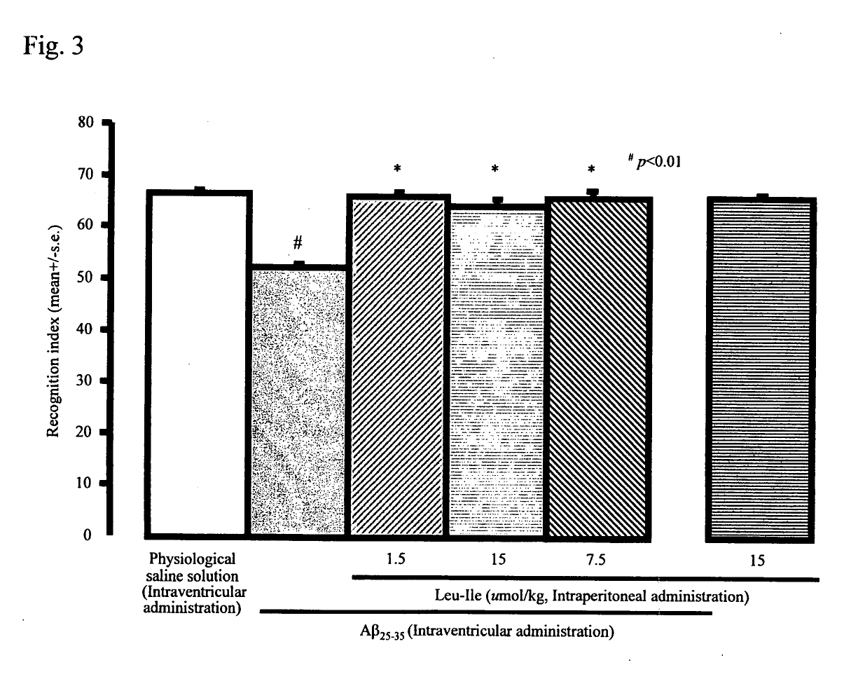

A novel object recognition test was conducted for each of the above groups 3 to 5 days after intraventricular administration of Aβ25-35 or a physiological saline solution. The test results on day 5 after administration of Aβ25-35 or a physiological saline solution were compared with the other test results on day 5 after administration (FIG. 3). Furthermore, Leu-Ile (15 μmol/Kg) was intraperitoneally administered to untreated mice and then the mice were subjected to a novel object recognition test similar to that conducted for the above test groups.

As shown in FIG. 3, the Alzheimer's disease model groups subjected to administration of Aβ25-35 exerted significantly decreased recognition indices compared with the control group to which only a physiological saline solution had been administered. However, decreased recognition indices were not observed in the Leu-Ile 1.5 μmol/Kg administrated group, the Leu-Ile 15 μmol/Kg administrated group, and the Leu-Ile 75 μmol/Kg administrated group. It was thus confirmed that Leu-Ile exerts effects of significantly inhibiting the progression of Alzheimer's dementia. In addition, dose dependence was not observed. When Leu-Ile (15 μmol/Kg) was administered to untreated mice, effects of improving memory were not observed.

As described in the results of Examples 1 to 3 above, it was confirmed that Leu-Ile has effects of preventing intracerebral protein oxidation due to deposition of amyloid protein that takes place associated with aging or the like. It was also confirmed that Leu-Ile inhibits reduced memory resulting from deposition of amyloid protein via oral or intraperitoneal administration of even a trace amount of Leu-Ile.

Furthermore, it was considered that the effects of Leu-Ile on the Alzheimer models are not due to activation of AKt based on the results of measurement of the influence and the effects on TNF-α mRNA expression in the hippocampal regions and the results of measurement of the influence and the effects on GDNF mRNA expression, which were conducted simultaneously with the above experiments.

### Preparation Example 1

### Tablet

Tablets with the following composition are prepared according to a conventional method.

Prescription:

| | | |
|---|---|---|
| Leu-Ile | 25 | mg |
| Lactose | 138.4 | mg |
| Potato starch | 30 | mg |
| Hydroxypropylcellulose | 6 | mg |
| Magnesium stearate | 0.6 | mg |
| Total | 200 | mg |

### Preparation Example 2

### Injection

An injection with the following composition is prepared according to a conventional method.

Prescription:

| | | |
|---|---|---|
| Leu-Ile | 2 | mg |
| D-mannitol | 10 | mg |

| | |
|---|---|
| Hydrochloric acid aqueous solution | Adequate dose |
| Sodium hydroxide aqueous solution | Adequate dose |
| Distilled water for injection | Adequate dose |

| | | |
|---|---|---|
| Total | 2.00 | mL |

### Preparation Example 3

### Dietary Supplement Tablet

Dietary supplement tablets with the following composition are prepared according to a conventional method.

Prescription:

| | | |
|---|---|---|
| Leu-Ile | 20 | mg |
| Erythritol | 245 | mg |
| Carboxymethylcellulose calcium | 5 | mg |
| Flavoring agent | 10 | mg |
| Sucrose fatty acid ester | 20 | mg |
| Total | 300 | mg |

All publications, patents, and patent applications cited in this specification are herein incorporated by reference in their entirety.

### INDUSTRIAL APPLICABILITY

The present invention is applicable as a pharmaceutical composition or a nutritional composition for dietary supplements for inhibiting intracerebral oxidation or for preventing or treating mental deterioration in the fields of medicine and foods.

1-9. (canceled)

10. A method for inhibiting intracerebral oxidation, which comprises administering a peptide consisting of Leu and Ile or a modified form thereof to a subject in need thereof.

11. The method for inhibiting intracerebral oxidation according to claim 10, wherein the peptide consisting of Leu and Ile is Leu-Ile.

12. The method for inhibiting intracerebral oxidation according to claim 10, wherein Leu-Ile is used.

13. The method for inhibiting intracerebral oxidation according to claim 10, wherein intracerebral oxidation takes place due to an increased amyloid protein level.

14. The method for inhibiting intracerebral oxidation according to claim 11, wherein intracerebral oxidation takes place due to an increased amyloid protein level.

15. The method for inhibiting intracerebral oxidation according to claim 12, wherein intracerebral oxidation takes place due to an increased amyloid protein level.

16. The method for inhibiting intracerebral oxidation according to claim 13, wherein intracerebral oxidation is the nitration of intracerebral protein.

17. The method for inhibiting intracerebral oxidation according to claim 14, wherein intracerebral oxidation is the nitration of intracerebral protein.

18. The method for inhibiting intracerebral oxidation according to claim 15, wherein intracerebral oxidation is the nitration of intracerebral protein.

19. A method for preventing or treating mental deterioration, which comprises administering a peptide consisting of Leu and Ile or a modified form thereof to a subject in need thereof.

20. The method for preventing or treating mental deterioration according to claim 19, wherein the peptide consisting of Leu and Ile is Leu-Ile.

21. The method for preventing or treating mental deterioration according to claim 19, wherein Leu-Ile is used.

22. The method for preventing or treating mental deterioration according to claim 19, wherein mental deterioration takes place due to an increased amyloid protein level.

23. The method for preventing or treating mental deterioration according to claim 20, wherein mental deterioration takes place due to an increased amyloid protein level.

24. The method for preventing or treating mental deterioration according to claim 21, wherein mental deterioration takes place due to an increased amyloid protein level.
